(19) United States
(12) Patent Application Publication
(10) Pub. No.: US 2019/0302080 A1
(43) Pub. Date: Oct. 3, 2019

(54) Food distinguishing apparatus for each individual health

(71) Applicant: Byungjoo Park, Cypress, CA (US)

(72) Inventor: Byungjoo Park, Cypress, CA (US)

(21) Appl. No.: 15/943,704

(22) Filed: Apr. 3, 2018

(51) Int. Cl. G01N 33/02; A61B 5/22

(52) U.S. Cl. CPC G01N 33/02; A61B 5/225

(57) ABSTRACT

A food distinguishing apparatus for each individual's healthy diet is disclosed. Because handgrip strength was reported to be one of the best future health predictors, having a good hand-grip strength could be a way to be healthy. The inventor has focused on a phenomenon of hand-grip strength changing by touching food. Touching good food immediately increases the muscle strength. The reactions with food vary on each individual. The invented apparatus measures the pressure variation of two clenched fingers' pinch-grip strength without touching food and with touching food under exerting pushing out force from an expandable container and distinguishes good food and bad food for each user.

### CLASSIFICATIONS

G01N33/02 Food

A61B5/225 Measuring muscular strength of the fingers

G01N33/00 Investigating or analyzing materials by specific methods not covered by the preceding groups

### FIELD OF THE INVENTION

This invention relates generally to an individual's food distinguishing apparatus based upon measuring the pressure variation of two clenched fingers' pinch-grip strength without and with touching food under exerting pushing out force from an expandable container.

### REFERENCES TO RELATED APPLICATIONS

[1] Abdominal Imaging, July/August 2004, Vol. 29 Issue 4, p 482-490.
[2] The American Journal of Clinical Nutrition, Volume 81, Issue 1, 1 Jan. 2005, Pages 79-86, https://doi.org/10.1093/ajcn/81.1.79
[3] Clinical Nutrition, Volume 33, Issue 1, February 2014, Pages 106-114, https://doi.org/10.1016/j.clnu.2013.03.003
[4] JAMA. 1999 Feb. 10; 281 (6):558-60
[5] U.S. Pat. No. 5,188,107A
[6] U.S. Pat. No. 9,782,624B2
U.S. Pat. Nos. 7,999,674B2, 5,412,560A, 8,249,946B2, 4,884,445A, 6,868,710B2

### BACKGROUND OF THE INVENTION

We are what we eat. Therefore, food is an important factor for our body. There have been many efforts to find answers on what HUMANS should eat. But the question should be changed to what EACH INDIVIDUAL should eat. Because we are all different.

How come somebody has a nut allergy and somebody does not? One reason could be the differences of organ volume configuration in each person. Based on the organ imaging study, the size of an organ volume differed up to 2 to 10 times in the normalized standard body.[1] The organ volume difference affects to the organ function. From this research, we can understand that the different organ volume configuration could make a different digestibility for the same food in each body.

We have no doubt animals can sense what they should eat. For example, cows do not hunt a rabbit to eat in nature. What about humans? To find the hidden sensor in humans, we focused on hand-grip strength as a predictor of nutrition status and a nutrition marker.[2, 3] Handgrip strength is known to be one of the best parameters to show current health condition and to predict future health condition.[4] Our conclusion from these studies is having a good hand-grip strength is a strategy to be healthy.

There is a phenomenon to change hand-grip strength by touching a problem area on the skin.[5] The phenomenon would be applied to change hand-grip strength by touching food. Food that increases hand grip strength could be considered good food. To avoid the food that makes hand-grip strength weak and to have the food that makes hand-grip strength strong might be the best diet habit. This invention introduces an apparatus to select food for each individual's healthy diet.

### SUMMARY OF THE INVENTION

Due to the existing differences of organ volume configurations, each individual's diet should be different like the animals' diet differences that relate on their organ structure differences. We invent this food distinguishing apparatus to determine good and bad food for each user.

The invented apparatus measures the pressure variation of two clenched fingers' pinch-grip strength under exerting pushing out force from an expandable container. The apparatus compares the measured each value of not touching food and touching food. If the value of touching food is greater than one without touching food, this means the food is good, which increases muscle strength. If the value of touching food is lesser than one without touching food, this means food is bad, which decreases muscle strength.

Most of grip strength tests use the compression test method. [7] On the contrary, the presented invention uses a tensile test method that measures a resistant force to push out the two clenched fingers from inside to outside as shown in FIG. 3a. The pressure measurement of resistance force, tensile test method, shows better sensitivity and higher accuracy than the compression test method. Other conventional compression test method makes the finger muscles easily tired and the aimed measurement from touching food, the size of the strength variation, is much smaller than the tensile test method.

### BRIEF DESCRIPTION OF THE DRAWINGS

The above and other objectives, features and other advantages of the present invention will be more clearly understood from the following detailed description taken in conjunction with the accompanying drawings, in which:

FIG. 1 is a schematic sketch of consisting parts of a food distinguishing apparatus;

FIG. 2 is a flowchart showing steps for food distinguishing by measuring the pressure variation of pinch-grip strength under exerting pushing out force from an expandable container;

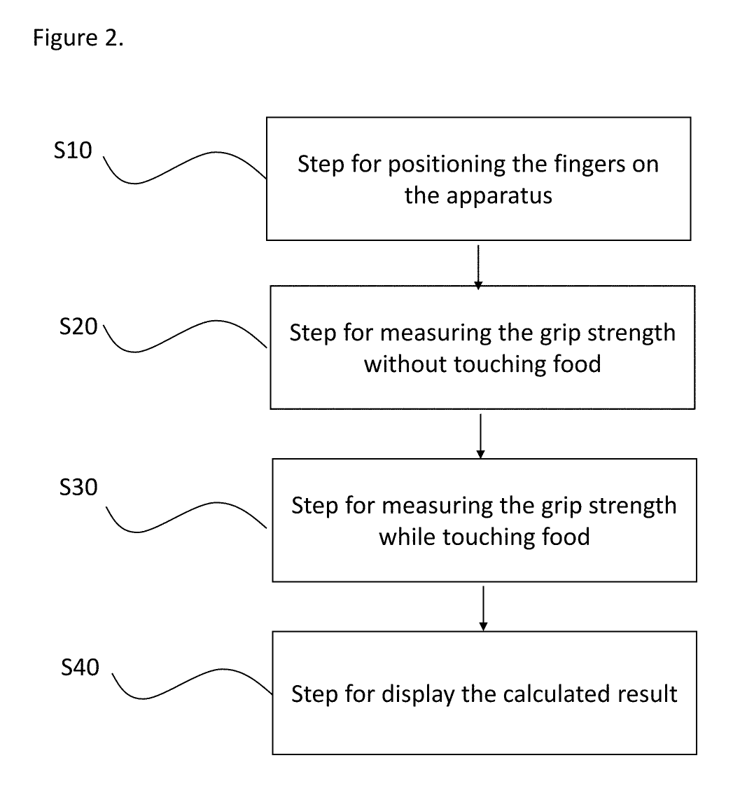

FIG. 3 is exemplary drawing showing the position of fingers for food distinguishing by measuring the pressure variation of two clenched fingers' strength according to the present inventions;

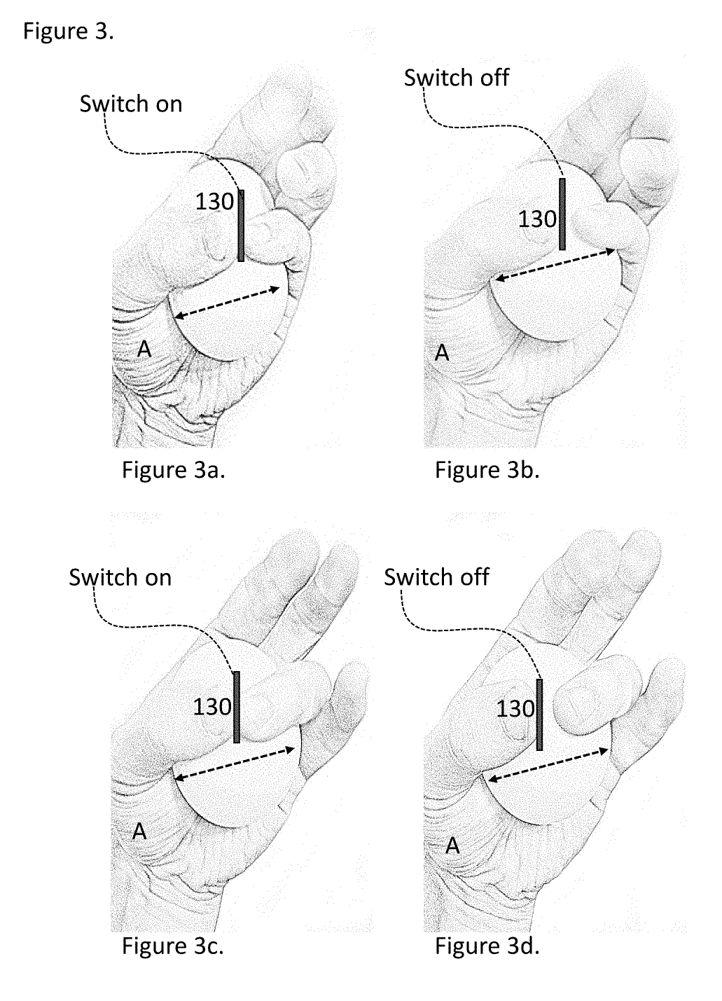

In FIG. 3a, clenched thumb and little finger makes switch turn on. In FIG. 3b, when the pressure reaches to the max of finger strength, the opened fingers makes switch turn off. FIG. 3c and FIG. 3d shows an example of clenched thumb and ring finger.

### REFERENCE TO DRAWING CODES

100: a computing part
110: an expandable container
120: an inflating part
130: a switch part
140: a pressure sensing part
150: a display part
A: hand

### DETAILED DESCRIPTION OF THE PREFERRED EMBODIMENTS

The steps of food distinguishing

1. Measure the two clenched fingers' pinch grip strength WITHOUT touching food—Measurement 1

(1) Make a circle with thumb and little finger and press the switch as shown in FIG. 3a.
A circle with thumb and ring finger, also possible as shown in FIG. 3c.
Usually the grip strength of a circle with thumb and middle finger or thumb and index finger is too strong to measure the changes. But not limited to use of middle finger and index finger.

(2) Try to clench the two fingers against the force of the apparatus.

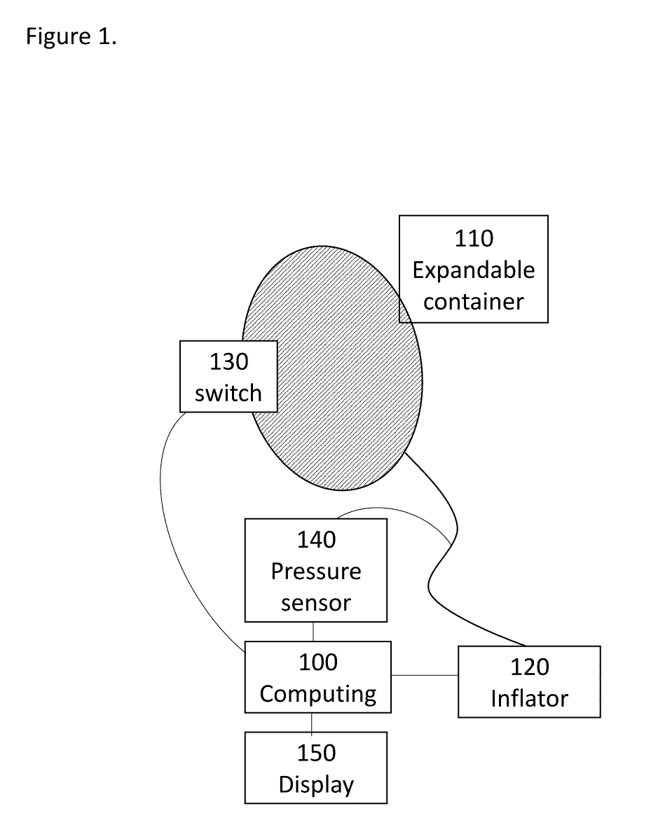

(3) When the fingers opened which turns switch off as shown in FIG. 1c, mark the pressure. This is measurement 1.

2. Measure the two clenched fingers' pinch grip strength WHILE touching food in other hand.—Measurement 2

(1) Use the same finger of the same hand combination that was applied in step 1.

For example, a circle made by left thumb and left little finger in Measurement 1, this time should be the same left thumb and left little finger.

(2) Do 1-(1) to 1-(3). However, this time it is while touching food with the other hand. This is measurement 2.

3. The food distinguishing apparatus calculates measurement 2−measurement 1.

If the value is positive, it is good food that increases muscle strength. If the value is negative, it is bad food that decreases muscle strength.

The examples of 110 in FIG. 1 is an expandable container that is any expandable shapes such as sphere, cylinder, cubic, etc. The expansion method includes, but is not limited to, inflating using an air pump, a hydraulic pump or an elongating rod to be extended with motor and gear power. The display of the result can be an image, sound and/or vibration.

Although the invention has been herein described in what is perceived to be the most practical and preferred embodiments, it is to be understood that the invention is not intended to be limited to the specific embodiments set above. Rather, it is recognized that modifications may be made by one of skill in the art of the invention without departing from the spirit or intent of the invention and, therefore, the invention is to be taken as including all reasonable equivalents to the subject matter of the appended claims and the description of the invention herein.

1. An apparatus for food distinguishing for each individual health comprising:
an expandable container which positioned inside circle of two clenched fingers;
a switch attached on the expandable container and clenched fingers makes switch turn on;
an inflator produces the pushing out force in the expandable container;
a pressure sensor measures the resisting force of the clenched fingers to the expanding of the container, when fingers opened, which makes switch turn off;
a computing part having a memory compares the pressure value of while touching food and the pressure value of without touching food. Touching food means placing food on any places of the skin such as the other side of the hand;
a display part prints good food by an image, sound and/or vibration, if the pressure value of while touching food is greater than the pressure value of without touching food, on the contrary, if the value of without touching food is greater than one of while touching food, the display part prints bad food.

2. The apparatus of claim 1, wherein the expandable container could be any expandable shapes such as sphere, cylinder, cubic, etc.

3. The apparatus of claim 1, wherein the inflator further comprises an air pump, a hydraulic pump or an elongating rod with motor and gear power.

4. The apparatus of claim 1, wherein the two clenched fingers further comprises thumb and little finger, thumb and ring finger, thumb and middle finger or thumb and index finger of any side of hands and he combination of two fingers must be the same in the two tests of without touching and while touching food.

5. The apparatus of claim 1, wherein the pressure value, if the value while touching food and without touching food is same, that means it is a neutral food.
